(19) United States
(12) Patent Application Publication
(10) Pub. No.: US 2006/0257340 A1
(43) Pub. Date: Nov. 16, 2006

(54) Formulation for skin-lightening agents

(76) Inventor: Xina Nair, St James, NY (US)

Correspondence Address: WARNER-LAMBERT COMPANY, 2800 PLYMOUTH RD, ANN ARBOR, MI, 48105, US

(21) Appl. No.: 10/544,090

(22) PCT Filed: Jan. 22, 2004
(86) PCT No.: PCT/IB04/00240

§ 371 Date: May 15, 2006

Related U.S. Application Data
(60) Provisional application No. 60/444,496, filed on Feb. 3, 2003.

(51) Int. Cl. A61K 8/34; A61K 8/33

(52) U.S. Cl. 424/62

(57) ABSTRACT

A new topical formulation for skin lightening agents, such as 4-cycloalkyl resorcinols, has been discovered. This new formulations enhances the efficacy of the skin-lightening agent. It allows the physician, or consumer, to utilize a lower dose of the skin-lightening agent (i.e. to utilize a lower concentration of the skin lightening agent). The formulation utilizes a carrier which contains a hydroxyl compound in admixture with a co-solvent.

### BACKGROUND OF THE INVENTION

1. Field of the Invention

The present invention is directed to a topical formulation suitable for administering depigmenting agents.

2. Summary of the Related Art

In humans, skin color (pigmentation) arises from a complex series of cellular processes that are carried out within a group of cells known as melanocytes. Melanocytes are located in the lower part of the epidermis and their function is to synthesize a pigment, melanin, which protects the body from the damaging effects of ultraviolet radiation.

The mechanism by which skin pigmentation is formed, melanogenesis, involves the following main steps: Tyrosine→L-Dopa→Dopaquinone Dopachrome→Melanin. The first two reactions in this series are catalyzed by the enzyme, tyrosinase. The activity of tyrosinase is promoted by the action of α-melanocyte stimulating hormone and UV rays.

Typically, melanogenesis leads to a darker skin tone (i.e. a tan). However, melanogenesis can lead to undesirable pigmentation patterns as well. Examples of such undesirable pigmentation include age spots, liver spots, etc. Such inappropriate pigmentation has lead to research to find compounds that will inhibit melanogenesis. One of the targets of this research is the inhibition of tyrosinase, the enzyme which catalyses the initial step in the generation of melanin.

U.S. Pat. No. 6,132,740 discloses a class of tyrosinase inhibitors that may be used as skin lighteners. All of the compounds of the '740 patent are 4-cycloalkyl resorcinols. The depigmenting activity of 4-cyclopentyl resorcinol and 4-cyclohexyl resorcinol is depicted in an animal model in the '740 patent (see column 10, lines 36-44). The formulation utilized for these tests was a 70:30 admixture of ethanol and propylene glycol with five (5) % of either 4-cyclohexyl resorcinol or 4-cyclopentyl resorcinol.

While the formulation of the '740 patent was efficacious as a skin lightening agent, there is always room for improvements. Such improvements include decreasing the amount of active agent that is required to achieve the therapeutic effect.

### SUMMARY OF THE INVENTION

In accordance with the present invention, a new topical formulation for skin lightening agents, such as 4-cycloalkyl resorcinols, has been discovered. This new formulation enhances the efficacy of the skin-lightening agent. It allows the physician, or consumer, to utilize a lower dose of the skin-lightening agent (i.e. to utilize a lower concentration of the skin lightening agent).

The new formulation contains a skin-lightening agent in combination with a carrier. The carrier contains at least one hydroxyl solvent and at least one co-solvent. Examples of suitable hydroxyl solvents include glycols, lower alkanols, or water. Examples of suitable co-solvents include a dianhydrohexitol, a cyclic amide, cyclic carbonate, cyclic carbamate, or a lower alkyl ester of a C₁₀-C₂₀ fatty acid.

Typically, the formulation will be a liquid. It will contain from about 0.1 to about 10% (wt/vol) of the skin-lightening agent. The formulation will also contain a carrier. The carrier may constitute up to 99.9% (vol/vol) of the formulation, but at a minimum will constitute at least 80% (vol/vol) of the formulation. The carrier will comprise from about about 40 to about 95% (vol/vol) of the hydroxyl solvent and from about 5 to about 40% (vol/vol) of the co-solvent. Often multiple hydroxyl solvents will be incorporated into the formulation. A glycol is typically used in combination with a lower alkalnol. The relevant quantity of glycol and lower alkanol can vary widely.

In a more specific embodiment, the skin-lightening agent is 4-cyclopentyl resorcinol or 4-cyclohexyl resorcinol, present in the quantity of 0.5 to 4% (wt/vol). The co-solvent is an isorbide present in the carrier in the quantity of about 10 to 20% (vol/vol). The remainder of the carrier will be composed of an admixture of glycol and alcohol.

The formulation, which is typically a liquid, is used in a conventional manner for dermatologicals. The patient will apply the formulation to the area of the skin requiring lightening. The formulation may be applied from 1 to 4 times daily until the patient achieves the desired pigmentation tone.

Typically, the formulation will be packaged for retail distribution. Thus a further embodiment of the invention is directed to a kit containing the formulation, packaged in a manner suitable for retail distribution, and labeled in a manner which instructs the patient, or health care provider, how to use the formulation in order to lighten their skin.

### BRIEF DESCRIPTION OF THE DRAWING

The invention is described with reference to the following figures, which are presented for the purpose of illustration only and which are not intended to be limiting of the invention.

FIG. 1 is a graphic representation comparing the depigmenting effect on Yucatan swine of topically applied 1% (wt/vol) 4-cyclopentylresorcinol and 2% (wt/vol) 4-cyclohexylresorcinol in HG/PG/DMI/EtOH with the HG/PG/DMI/EtOH solution alone. The asterisks denote statistical significance from the solution alone using ANOVA and Kruskal-Wallis test (p<0.05).

FIG. 2 is a graphic representation comparing the reversibility of depigmentation after treatment discontinuation for the HG/PG/DMI/EtOH solution alone to different concentrations of 4-cyclopentylresorcinol and 4-cyclohexylresorcinol in the solution.

FIG. 3A is a graphic representation comparing the effect on the skin pigmentation of Yucatan swine of topical administration of 1% (wt/vol) and 2% (wt/vol) 4-cyclopentylresorcinol in PG/EtOH. An asterisk denotes statistical difference from the respective vehicle controls.

FIG. 3B is a graphic representation comparing the effect on the skin pigmentation of Yucatan swine of topical administration of 1% (wt/vol) and 2% (wt/vol) 4-cyclopentylresorcinol in HG/PG/DMI/EtOH to 2% (wt/vol) 4-cyclopentylresorcinol in PG/EtOH and to the HG/PG/DMI/EtOH vehicle. An asterisk denotes statistical difference from the vehicle control.

FIG. 3C is a graphic representation comparing the effect on the skin pigmentation of Yucatan swine of topical administration of 1% (wt/vol) and 2% (wt/vol) 4-cyclopentylresorcinol in HG/PG/DMI/EtOH/IPM to 2% (wt/vol) 4-cyclopentylresorcinol in PG/EtOH and to the HG/PG/DMI/EtOH plus 2% (wt/wt) IPM vehicle alone. An asterisk denotes statistical difference from the vehicle control.

FIG. 4A is a graphic representation showing the effect on the skin pigmentation of Yucatan swine of topical administration of 5% (wt/vol) 4-cyclohexylresorcinol in PG/EtOH.

FIG. 4B is a graphic representation comparing the effect on the skin pigmentation of Yucatan swine of topical administration of 2% (wt/vol) 4-cyclohexylresorcinol in HG/PG/DMI/EtOH/IPM to the HG/PG/DMI/EtOH plus 2% (wt/wt) IPM solution alone and to 5% (wt/vol) 4-cyclohexylresorcinol in PG/EtOH. An asterisk denotes statistical difference from the vehicle control.

FIG. 4C is a graphic representation comparing the effect on the skin pigmentation of Yucatan swine of topical administration of 2% (wt/vol) 4-cyclohexylresorcinol in HG/PG/DMI/EtOH to the HG/PG/DM/EtOH solution alone. An asterisk denotes statistical difference from the vehicle control.

FIG. 5A is a graphic representation comparing the effect on the skin pigmentation of Yucatan swine of topical administration of 1% (wt/vol) and 2% (wt/vol) 4-cyclopentylresorcinol in an HG/PG/DM/EtOH/IPM vehicle and 2% (wt/vol) 4-cyclohexylresorcinol in an HG/PG/DM/EtOH/IPM vehicle to the HG/PG/DMI/EtOH plus 2% (wt/wt) IPM vehicle alone. An asterisk denotes statistical difference from the vehicle control.

FIG. 5B is a graphic representation comparing the effect on the skin pigmentation of Yucatan swine of topical administration of 1% (wt/vol) and 2% (wt/vol) 4-cyclopentylresorcinol in a HG/PG/DMI/EtOH vehicle and 2% (wt/vol) 4-cyclohexylresorcinol in the HG/PG/DMI/EtOH vehicle to the HG/PG/DMI/EtOH vehicle alone. An asterisk denotes statistical difference from the vehicle control.

### DETAILED DESCRIPTION

The headings below have been added to facilitate the review of this document by the reader. They should not be construed as limiting the disclosure or claims in any manner.

A) Definitions

The issued U.S. patents, published patent applications, and references that are cited herein are hereby incorporated by reference to the same extent as if each were specifically and individually indicated to be incorporated by reference. Any inconsistency between these publications and the present disclosure shall be resolved in favor of the present disclosure.

The following terms are used herein according to the following definitions:

As used herein, the term “comprises” means “includes, but is not limited to.”

The term “topical” or “topically” as used herein refers to the application of a formulation externally to the skin or a portion of the external surface of the body.

As used herein, the term “skin-lightening agent” encompasses an agent that increases the degree to which the skin, or a portion of the external surface of the body, is pale or white in color. Such an agent decreases the degree to which color or pigmentation is present or formed within the skin or a portion of the external surface of the body. In some embodiments, the effect achieved by a skin-lightening agent is achieved by preventing the formation of skin pigmentation. As used herein, this term is equivalent to the terms “active agent,” “pigmentation-inhibiting agent,” “lightening agent,” “depigmenting agent” and “depigmentation agent.”

The term “admixture” means two or more components mixed together resulting in a combination of the components. By way of nonlimiting example, one component is dissolved in another component.

The term “carrier” is used herein to refer to a pharmaceutically acceptable vehicle as described herein for the skin-lightening agent and/or other pharmacologically active agent. The carrier facilitates delivery of the skin-lightening agent to the target site in the skin of a subject without terminating the skin-lightening function of the agent. Any pharmaceutical excipient incorporated into the formulation should be considered part of the carrier.

The term “hydroxyl compound” refers to any compound having at least one hydroxyl group. For purposes of the invention, preferred hydroxyl compounds include, without limitation, glycols, lower alkanols, and water. Hydroxyl compound and hydroxyl solvent should be considered as synonoms.

The term “glycol” refers to a straight or branched chain hydrocarbon having at least two hydroxyl substituents. In some embodiments, the glycol has from about two to about twelve carbon atoms (C₂-C₁₂). As used herein, the term “glycol” also encompasses polymeric versions of the glycols described herein. For example, reference to a “C₂ glycol” is intended to include both ethylene glycol and polyethylene glycol.

The term “co-solvent” as used herein refers to a compound in which the skin-lightening agent is soluble. In some embodiments the co-solvent also serves additional functions, such as enhancing the activity of the skin-lightening agent.

A “fatty acid” as used herein refers to a long hydrocarbon chain that has a carboxyl group at one end. The hydrocarbon chain optionally includes one or more carbon-carbon double bonds.

As used herein, the term “derivative” means a compound that retains the underlying chemical structure of the original compound, but has been structurally modified on one or more functional groups. The term “derivative” includes, but is not limited to, compounds that can be readily converted into the original compound upon administration in vivo, e.g., prodrug forms of the original compound.

As used herein, the term “analog” refers to a compound that retains the same core structure as the compound referred to, but is optionally structurally modified (such as by addition or substitution). The term “analog” is intended to encompass the core structure compound, as well as compounds with modified structures.

By way of example, the term “resorcinol analog” includes resorcinol (1,3-benzenediol) and substituted resorcinols, as defined herein. In some embodiments, the resorcinol analog is a 4-substituted resorcinol. As used herein, the term “resorcinol derivative” refers to a resorcinol analog wherein one or more hydroxyl groups of the resorcinol are derivatized, e.g., as ester, carbonate, carbamate, or ether derivatives. In some embodiments, the resorcinol derivative acts as a prodrug, which can be readily converted into the original resorcinol compound upon administration of the compound in vivo, such as upon topical application to the skin, such that the resorcinol derivative ultimately produces its biological (e.g., skin-lightening) effect. As used herein, reference to “a resorcinol” or “resorcinol compound” encompasses resorcinol analogs and resorcinol derivatives, as defined herein.

The term “pharmaceutically acceptable” is used herein to mean suitable for use in mammals. Pharmaceutically acceptable salts of a compound include acid and base addition salts thereof. Suitable acid addition salts are formed from acids that form non-toxic salts. Nonlimiting examples include hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate (i.e., 1,1-methylene-bis-(2-hydroxy-3-naphthoate)) salts. Suitable base salts are formed from bases which form non-toxic salts. Nonlimiting examples include aluminum, sodium, potassium, calcium, magnesium, zinc and diethanolammonium salts. For a review of suitable salts, see, e.g., Berge et al, J. Pharm. Sci. 66:1-19 (1977) and Remington: The Science and Practice of Pharmacy, 20th Ed., ed. A. Gennaro, Lippincott Williams & Wilkins, 2000.

Pharmaceutically acceptable ester derivatives include those esters that retain, upon hydrolysis of the ester bond, the biological effectiveness and properties of the active carboxylic acid or alcohol component, and do not produce undesirable biological effects. For a description of pharmaceutically acceptable esters as prodrugs, see Bundgaard, E., ed., Design of Prodrugs, Elsevier Science Publishers, Amsterdam, 1985. Generally, ester formation is accomplished via conventional synthetic techniques. (See, e.g., March's Advanced Organic Chemistry, 3^{rd} Ed., John Wiley & Sons, New York p. 1157, 1985 and references cited therein, and Mark, et al., Encyclopedia of Chemical Technology, John Wiley & Sons, New York, 1980.) Also contemplated within the scope of the invention are formulation components that individually comprise both a pharmaceutically acceptable salt moiety and a pharmaceutically acceptable ester moiety.

The term “lower alkanol” is intended to encompass all C₁-C₆ alkanols, including, without limitation, all straight chain or branched lower alkanols. These compounds may contain chiral centers and therefore may exist in different enantiomeric and diastereomeric forms. Any optical isomers, any regioisomers, and any stereoisomers of these compounds and mixtures thereof, can be used as the active agent in the formulation of the invention.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

The term “substituted” is used herein to refer to the addition of a functional group at a location on a compound. It means that one or more hydrogens of the designated moiety are replaced, provided that no atom's normal valency is exceeded, and provided that the substitution results in a stable compound. A variety of locations may be substituted or the location may be specified. The functional group may include one or more atoms and may be added in one reaction or using several reactions.

The terms “stable compound” and “stable structure” refer to a compound that is sufficiently robust to survive isolation to a useful degree of purity from a reaction mixture and formulation into an efficacious agent.

The term “alkyl”, as used herein, unless otherwise indicated, includes saturated monovalent hydrocarbon radicals having straight or branched moieties or combinations thereof. In some embodiments the alkyl has from 1-12 carbons. In some embodiments the alkyl has from 1-8 carbons. In some embodiments the alkyl has from 1-6 carbons. The term “lower alkyl” refers to an alkyl moiety containing from 1-4 carbon atoms.

The term “cycloalkyl” as employed herein, unless otherwise indicated, includes saturated cyclic hydrocarbon groups having from 3 to about 12 carbons. In some embodiments the cycloalkyl has from 3 to about 8 carbons. In some embodiments the cycloalkyl has from 3 to about 6 carbons. Cycloalkyl groups include, without limitation, cyclopropyl, cyclobutyl, cyclopentyl, cyclohexyl, cycloheptyl, and cyclooctyl.

The term “alkenyl”, as used herein, unless otherwise indicated, includes unsaturated hydrocarbon radicals having straight or branched moieties or combinations thereof, with one or more double bonds. In some embodiments the alkenyl has one or two double bonds. In some embodiments the alkenyl has from 2-12 carbons. In some embodiments the alkenyl has from 2-8 carbons. In some embodiments the alkenyl has from 2-6 carbons.

The term “cycloalkenyl” as employed herein, unless otherwise indicated, includes partially unsaturated cyclic hydrocarbon groups having from 3 to about 12 carbons. In some embodiments the cycloalkenyl has from 3 to about 8 carbons. In some embodiments the cycloalkenyl has from 5 to about 6 carbons. Cycloalkenyl groups include, without limitation, cyclopentenyl and cyclohexenyl.

The term “aryl,” as used herein, unless otherwise indicated, refers to phenyl or naphthyl optionally substituted with one or more substituents independently selected from halogen, (C₁-C₆)alkyl, (C₁-C₆)alkoxy, amino, (C₁-C₆)alkylamino, di-[(C₁-C₆)alkyl)]amino, nitro, cyano and trifluoromethyl. In some embodiments the aryl group is substituted with from zero to two substituents.

The term “acyl,” as used herein, unless otherwise indicated, includes a radical of the general formula RCO wherein R is alkyl, alkoxy, aryl, arylalkyl, or arylalkyloxy and the terms “alkyl” or “aryl” are as defined above.

The term “acyloxy”, as used herein, unless otherwise indicated, includes O-acyl groups wherein “acyl” is as defined above.

The term “heteroaryl”, as used herein, unless otherwise indicated, refers to (C₂-C₉)heteroaryl containing one to five N, O or S atoms. In some embodiments the heteroaryl is a 5- or 6-membered heteroaryl. In some embodiments, the heteroaryl is selected from furyl, thienyl, thiazolyl, pyrazolyl, isothiazolyl, oxazolyl, isoxazolyl, pyrrolyl, triazolyl, tetrazolyl, imidazolyl, 1,3,5-oxadiazolyl, 1,2,4-oxadiazolyl, 1,2,3-oxadiazolyl, 1,3,5-thiadiazolyl, 1,2,3-thiadiazolyl, 1,2,4-thiadiazolyl, pyridyl, pyrimidinyl, pyrazinyl, pyridazinyl, 1,2,4-triazinyl, 1,2,3-triazinyl, 1,3,5-triazinyl, pyrazolo[3,4-b]pyridinyl, cinnolinyl, pteridinyl, purinyl, 6,7-dihydro-5H-[1]pyridinyl, benzo[b]thiophenyl, 5,6,7,8-tetrahydro-quinolin-3-yl, benzoxazolyl, benzothiazolyl, benzisothiazolyl, benzisoxazolyl, benzimidazolyl, thianaphtenyl, isothianaphthenyl, benzofuranyl, isobenzofuranyl, isoindolyl, indolyl, indolinzinyl, indazolyl, isoquinolinyl, quinolinyl, phthalazinyl, quinoxalinyl, quinazolinyl, and benzoxazinyl. One of ordinary skill in the art will understand that the connection of said (C₂-C₉)heteroaryl ring can be through a carbon atom or through a nitrogen heteroatom where possible.

The term “heterocycloalkyl,” as used herein, refers to a (C₂-C₉)heterocycloalkyl containing one to five N, O or S atoms. In some embodiments the heterocycloalkyl is a 5- or 6-membered heterocycloalkyl. In a preferred embodiment, the heterocycloalkyl group is selected from pyrrolidinyl, tetrahydrofuranyl, dihydrofuranyl, tetrahydropyranyl, pyranyl, thiopyranyl, aziridinyl, oxiranyl, methylenedioxyl, chromenyl, isoxazolidinyl, 1,3-oxazolidin-3-yl, isothiazolidinyl, 1,3-thiazolidin-3-yl, 1-2-pyrazolidin-2-yl, 1,3-pyrazolidin-1-yl, piperidinyl, thiomorpholinyl, 1,2-tetrahydrothiazin-2-yl, 1,3-tetrahydrothiazin-3-yl, tetrahydrothiadiazinyl, morpholinyl, 1,2-tetrahydrodiazin-2-yl, 1,3-tetrahydrodiazin-1-yl, tetrahydroazepinyl, piperazinyl, and chromanyl. One of ordinary skill in the art will understand that the connection of the heterocycloalkyl ring can be through a carbon atom or through a nitrogen heteroatom where possible.

The term “one or more substituents”, as used herein, refers to a number of substituents that equals from one to the maximum number of substituents possible based on the number of available bonding sites.

The term “halogen”, as used herein, unless otherwise indicated, refers to chlorine, fluorine, bromine and iodine.

The term “effective” is used herein to indicate that the active agent is administered in an amount and at an interval that results in the desired treatment or improvement in the disorder or condition being treated (e.g., an amount effective to decrease pigmentation or ameliorate hyperpigmentation). This term is intended to include treatments for cosmetic purposes (e.g., cosmetically effective amounts). Similarly, the term “pharmaceutical” as used herein is intended to encompass cosmetic purposes.

B) Skin-Lightening Agents

A useful active agent is a melanin-synthesis inhibitor. A nonlimiting example of a melanin-synthesis inhibitor is a tyrosinase inhibitor. Typically, the tyrosinase inhibitor will be one of the 4-substituted resorcinol derivatives as described by Formula I-XI below.

The resorcinol compounds for use in the formulations of the invention are selected from any of the resorcinol analogs and derivatives thereof currently known or described in the art, or to be described in the future, and combinations thereof. Examples of resorcinol analogs and derivatives thereof include, but are not limited to, those described in U.S. Pat. No. 4,959,393 to Torihara et al. (issued Sep. 25, 1990), describing the use of 4-n-butylresorcinol, 4-isoamylresorcinol and other resorcinol analogs; U.S. Pat. No. 6,132,740 to Hu (issued Oct. 17, 2000), describing the use of various resorcinol analogs; WO 00/56702 by Pfizer Inc. (published Sep. 28, 2000), describing various resorcinol analogs; WO 00/56279 by Pfizer Inc. (published Sep. 28, 2000), describing 4-(2,4-dihydroxyphenyl)cyclohexanol; WO 02/20474 by Pfizer Products, Inc. (published Mar. 14, 2002), describing various resorcinol analogs; WO 02/24613 by Pfizer Products, Inc. (published Mar. 28, 2002), describing various resorcinol analogs; and EP 0904774 by Pfizer Products Inc. (published Mar. 31, 1999, describing various resorcinol analogs; and other patents and patent publications describing resorcinol analogs for skin-lightening.

In some embodiments the resorcinol analog is a substituted resorcinol such as a 4-substituted resorcinol or a pharmaceutically acceptable salt or derivative thereof. In some embodiments, the skin-lightening agent is a resorcinol analog (such as a tyrosinase inhibitor) described by the structure of any one of formulas I to XI. Various 4-substituted resorcinols have been described in the art as described above. These publications describe, inter alia, 4-substituted resorcinol compounds of the formula (I):

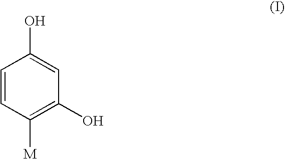

In some embodiments, the variable M is a straight or branched chain alkyl group. A nonlimiting example of such a 4-alkylresorcinol is 4-n-butylresorcinol. In some embodiments the alkyl group is further substituted. A nonlimiting example of such a further substitution includes the addition of a carboxyl group at the terminus of the alkyl chain.

In some other embodiments, the variable M is a cyclic group. In some such embodiments, the 4-substituted resorcinol is a compound of formula II:

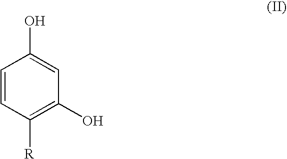

or a pharmaceutically acceptable salt or derivative thereof, wherein:

R is a (C₃-C₈)cycloalkyl ring or (C₅-C₈)cycloalkenyl ring, wherein either the cycloalkyl ring or cycloalkenyl ring is substituted by one to three substituents independently selected from the group consisting of cyano; halo; (C₁-C₆)alkyl; aryl; (C₂-C₉)heterocycloalkyl; (C₂-C₉)heteroaryl; aryl(C₁-C₆)alkyl-; ═O; ═CHO(C₁-C₆)alkyl; amino; hydroxy; (C₁-C₆)alkoxy; aryl(C₁-C₆)alkoxy-; (C₁-C₆)acyl; (C₁-C₆)alkylamino-; aryl(C₁-C₆)alkylamino-; amino(C₁-C₆)alkyl-; (C₁-C₆)alkoxy-CO—NH—; (C₁-C₆)alkylamino-CO—; (C₂-C₆)alkenyl; (C₂-C₆)alkynyl; hydroxy(C₁-C₆)alkyl-; (C₁-C₆)alkoxy(C₁-C₆)alkyl-; (C₁-C₆)acyloxy(C₁-C₆)alkyl-; nitro; cyano(C₁-C₆)alkyl-; halo(C₁-C₆)alkyl-; nitro(C₁-C₆)alkyl-; trifluoromethyl; trifluoromethyl(C₁-C₆)alkyl-; (C₁-C₆)acylamino-; (C₁-C₆)acylamino(C₁-C₆)alkyl-; (C₁-C₆)alkoxy(C₁-C₆)acylamino-; amino(C₁-C₆)acyl-; amino(C₁-C₆)acyl(C₁-C₆)alkyl-; (C₁-C₆)alkylamino(C₁-C₆)acyl-; ((C₁-C₆)alkyl)₂amino(C₁-C₆)acyl-; —CO₂R²; —(C₁-C₆)alkyl-CO₂R²; —C(O)N(R²)₂; —(C₁-C₆)alkyl-C(O)N(R²)₂; R²ON═; R²ON═(C₁-C₆)alkyl-; R²ON═CR²(C₁-C₆)alkyl-; —NR²(OR²); —(C₁-C₆)alkyl-NR²(OR²); —C(O)(NR²OR²); —(C₁-C₆)alkyl-C(O)NR²OR²); —S(O)ₘR²; wherein each R² is independently selected from hydrogen, (C₁-C₆)alkyl, aryl, or aryl(C₁-C₆)alkyl-; R³C(O)O—, wherein R³ is (C₁-C₆)alkyl, aryl or aryl(C₁-C₆)alkyl-; R³C(O)O—(C₁-C₆)alkyl-; R⁴R⁵N—C(O)—O—; R⁴R⁵NS(O)₂—; R⁴R⁵NS(O)₂(C₁-C₆)alkyl-; R⁴S(O)₂R⁵N—; R⁴S(O)₂R⁵N(C₁-C₆)alkyl-; wherein m is 0, 1 or 2, and R⁴ and R⁵ are each independently selected from hydrogen or (C₁-C₆)alkyl; —C(═NR⁶)(N(R⁴)₂); or —(C₁-C₆)alkyl-C(═NR⁶)(N(R⁴)₂) wherein R⁶ represents OR² or R² wherein R² is defined as above;

with the proviso that the cycloalkenyl ring is not aromatic;

with the proviso that R must be substituted by at least one of R³(C(O)O—; R³(C(O)O—(C₁-C₆)alkyl-, R²ON═, R²ON═(C₁-C₆)alkyl-, R²ON═CR²(C₁-C₆)alkyl-, —NR²(OR²), R⁴R⁵NS(O)₂—; R⁴R⁵NS(O)₂(C₁-C₆)alkyl-, R⁴S(O)₂R⁵N—, or R⁴S(O)₂R⁵N(C₁-C₆)alkyl-; and

with the proviso that when R is only substituted by one of R²ON═, then R² cannot be hydrogen.

Such compounds are described in e.g., WO 00/56702 by Pfizer Inc. (published Sep. 28, 2000). A nonlimiting example of such a compound is 4-(2,4-dihydroxyphenyl)cyclohexanol, which is described in more detail in, e.g., WO 00/56279 by Pfizer Inc. (published Sep. 28, 2000).

In some embodiments, the 4-substituted resorcinol is a compound of formula III,

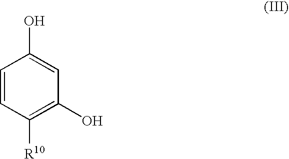

or a pharmaceutically acceptable salt or derivative thereof, wherein:

R¹⁰ is a (C₃-C₈)cycloalkyl or (C₅-C₈)cycloalkenyl ring substituted by —N(R¹¹)CONR¹²R¹³ wherein R¹¹ and R¹² are independently selected from hydrogen, (C₁-C₆)alkyl, and aryl(C₁-C₆)alkyl, and R¹³ is hydrogen, (C₁-C₆)alkyl, aryl(C₁-C₆)alkyl, or aryl; —N(R¹⁴)COR¹⁵ wherein R¹⁴ is hydrogen, (C₁-C₆)alkyl, aryl(C₁-C₆)alkyl-, or OH, and R¹⁵ is (C₇-C₁₀)alkyl, aryl, aryl(C₁-C₆)alkyl-, —O-aryl, CF₃, heterocycloalkyl, —(C₁-C₆)alkylheterocycloalkyl, —(C₂-C₇)alkenylheterocycloalkyl, heteroaryl, —(C₁-C₆)alkyl heteroaryl, —(C₂-C₇)alkenylheteroaryl, —(C₂-C₇)alkenylaryl, —(C₂-C₇)alkenylCOaryl, —(C₁-C₆)alkylN(R¹⁴)CO-aryl, —(C₁-C₆)alkylCO-aryl, —(C₁-C₆)alkylhydroxyaryl, —(C₁-C₆)alkyl-X-aryl, (C₂-C₇)alkenyl, benzyhydryl, 5-hydroxyoxoindanyl, or tetrahydronaphthalenyl, wherein X is O, S, SO, SO₂ or NR¹¹; —N(R¹¹)OCOaryl; ═CHCO₂R¹¹; ═CHCONR¹¹R¹²; ═CHCN; ═NNHSO₂R¹⁶ wherein R¹⁶ is aryl; —N(O)═CHR¹⁶; —OC(O)ONR¹¹R¹⁷ wherein R¹⁷ is aryl, aryl(C₁-C₆)alkyl-, —(C₁-C₆)alkylCO₂(C₁-C₆)alkyl, —CO₂(C₁-C₆)alkyl,

—CO₂aryl, or —CO₂(C₁-C₆)alkylaryl; amino(C₁-C₆)alkylarylCO₂—; or —OC(O)OR¹⁸ wherein R¹⁸ is (C₁-C₆)alkyl, aryl(C₁-C₆)alkyl, or aryl;

with the proviso that the cycloalkenyl ring is not aromatic.

Such compounds are described in more detail in e.g., WO 02/24613 by Pfizer Products, Inc. (published Mar. 28, 2002).

In some embodiments, the 4-substituted resorcinol is a compound of formula IV;

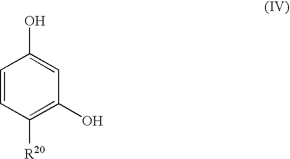

wherein R²⁰ is a (C₃-C₈)cycloalkyl or (C₅-C₈)cycloalkenyl ring substituted by ═CH₂, or a pharmaceutically acceptable salt or derivative thereof; with the proviso that the cycloalkenyl ring is not aromatic.

Such compounds are described in more detail in e.g., WO 02/24613 by Pfizer Products, Inc. (published Mar. 28, 2002).

In some embodiments, the 4-substituted resorcinol is a compound of formula V,

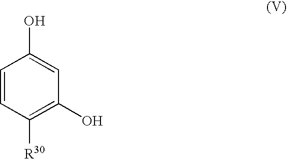

wherein R³⁰ is 3-cyclohexenyl, which is preferably unsubstituted, or a pharmaceutically acceptable salt or derivative thereof.

Such compounds are described in more detail in e.g., WO 02/24613 by Pfizer Products, Inc. (published Mar. 28, 2002).

In some embodiments, the 4-substituted resorcinol is a compound of formula VI,

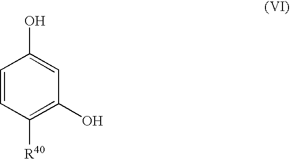

wherein R¹⁰ is a (C₃-C₈)cycloalkyl or (C₅-C₈)cycloalkenyl ring, wherein one of the carbon atoms of said cycloalkyl or cycloalkenyl rings is substituted by two groups such that the groups are taken together with the carbon to which they are attached to form a ring of the formula:

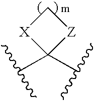

wherein X is O, S, SO, SO₂, or NR¹¹, wherein R¹¹ is hydrogen, (C₁-C₆)alkyl or aryl(C₁-C₆)alkyl; Z is CH₂, O, S, SO, or SO₂; m is 0-3; with the proviso that when m=0, then Z is CH₂; and with the proviso that the cycloalkenyl ring is not aromatic; or a pharmaceutically acceptable salt or derivative thereof.

Such compounds are described in more detail in e.g., WO 02/24613 by Pfizer Products, Inc. (published Mar. 28, 2002).

In some embodiments, the 4-substituted resorcinol is a compound of formula VII,

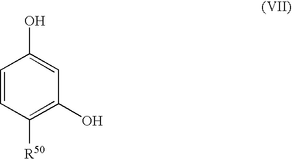

wherein R⁵⁰ is a (C₃-C₈)cycloalkyl or (C₅-C₈)cycloalkenyl ring, wherein one of the carbon atoms of said cycloalkyl or cycloalkenyl rings is substituted by two groups such that the groups are taken together with the carbon to which they are attached to form a ring of the formula:

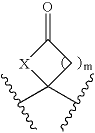

wherein X is O, S, SO, SO₂ or NR¹¹, wherein R¹¹ is hydrogen, (C₁-C₆)alkyl or aryl(C₁-C₆)alkyl; and m is 0-3; and with the proviso that the cycloalkenyl ring is not aromatic; or a pharmaceutically acceptable salt or derivative thereof.

Such compounds are described in more detail in e.g., WO 02/24613 by Pfizer Products, Inc. (published Mar. 28, 2002).

In some embodiments, the 4-substituted resorcinol is a compound of formula VIII,

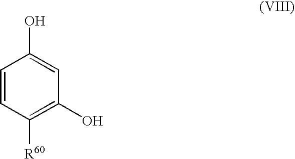

or a pharmaceutically acceptable salt or derivative thereof, wherein:

R⁶⁰ is a (C₃-C₈)cycloalkyl ring or (C₅-C₈)cycloalkenyl ring wherein either the cycloalkyl ring or cycloalkenyl ring is substituted by one of —N(R⁶¹)SO₂(CHR⁶¹)ₙR⁶² or —(C₁-C₆)alkylN(R⁶¹)SO₂(CHR⁶¹)ₙR⁶², wherein each R⁶¹ is independently selected from hydrogen, (C₁-C₆)alkyl, phenyl and benzyl; R⁶² is aryl, heteroaryl or heterocycloalkyl optionally substituted with one or more substituents independently selected from halogen, OH, —(C₁-C₆)alkyl, —(C₁-C₆)alkoxy, trifluoromethoxy, —S(O)ₘ(C₁-C₆)alkyl, amino, —N(R⁶¹)CO(C₁-C₆)alkyl, COOR⁶¹, —(C₁-C₆)alkylCOOR⁶¹, —CO(C₁-C₆)alkyl, —(C₁-C₆)alkylOH, —(C₁-C₆)alkylamino, di-((C₁-C₆)alkyl)amino, nitro, cyano, —CONH(CHR⁶¹)ₙCO₂R⁶¹, —CONR⁶¹N(R⁶¹)₂, trifluoromethyl, aryl, heteroaryl, and heterocycloalkyl; n is an integer from 0 to 6; and m is an integer from 0 to 2;

with the proviso that the cycloalkenyl ring is not aromatic.

Such compounds are described in more detail in e.g., WO 02/20474 by Pfizer Products Inc. (published Mar. 14, 2002).

In some embodiments, the 4-substituted resorcinol is a 4-cycloalkylresorcinol of formula (IX):

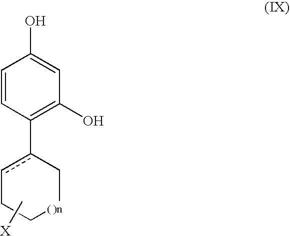

or a pharmaceutically acceptable salt or derivative thereof, wherein X is selected from the group consisting of hydrogen; OR⁷¹, wherein R⁷¹ represents hydrogen, (C₁-C₆)alkyl or aryl-(C₁-C₆)alkyl; OCOR⁷² wherein R⁷² represents (C₁-C₆)alkyl, aryl-(C₁-C₆)alkyl or aryl; halogen; (C₁-C₆)alkyl; aryl-(C₁-C₆)alkyl; SR⁷³ wherein R⁷³ represents hydrogen, (C₁-C₆)alkyl, or aryl-(C₁-C₆)alkyl; and NHR⁷¹ wherein R⁷¹ is defined as above;

wherein any of said aryl groups optionally can be substituted by zero to two substituents selected from the group consisting of halogen, (C₁-C₆)alkyl, (C₁-C₆)alkoxy, amino, (C₁-C₆)alkylamino, di-[(C₁-C₆)alkyl]amino, nitro, cyano, and trifluoromethyl;

n is 0 to 3; and

the dashed line indicates an optional double bond at that position.

Such 4-substituted resorcinols are described in, e.g., U.S. Pat. No. 6,132,740 to Hu (issued Oct. 17, 2000).

In some embodiments, the 4-cycloalkyl resorcinol is 4-cyclopentylresorcinol (formula (X)) or a pharmaceutically acceptable salt or derivative thereof:

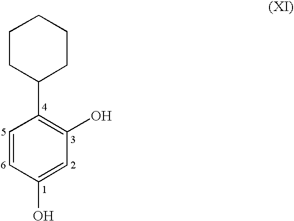

In some embodiments, the 4-cycloalkylresorcinol is 4-cyclohexylresorcinol (formula (XI)) or a pharmaceutically acceptable salt or derivative thereof:

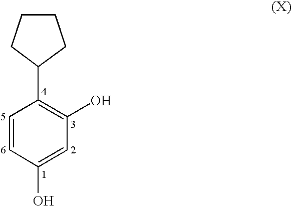

In some embodiments, these 4-cycloalkylresorcinols are further substituted on the cycloalkyl substituent.

In some embodiments, such substitution is at the 3′ position of the cyclopentyl ring or at the 3′ or 4′ position of the cyclohexyl ring.

The resorcinol compounds described herein may contain chiral centers and therefore may exist in different enantiomeric and diastereomeric forms. Any optical isomers and any stereoisomers of these compounds and mixtures thereof, can be used as the active agent in the formulation of the invention.

The resorcinol compounds also include compounds identical to those described and depicted but for the fact that one or more hydrogen, carbon or other atoms are replaced by isotopes thereof. In some embodiments such compounds are useful as research and diagnostic tools in metabolism and pharmacokinetic studies, and in binding assays.

Pharmaceutically acceptable acid and base addition salts of resorcinol compounds are also useful and can be made, e.g., as described in U.S. Pat. No. 6,132,740 to Hu (issued Oct. 17, 2000). The acids which are used to prepare the pharmaceutically acceptable acid addition salts of the aforementioned base compounds of this invention are those which form non-toxic acid addition salts. Nonlimiting examples of such salts include salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate (i.e., 1,1-methylene-bis-(2-hydroxy-3-naphthoate)) salts.

Those resorcinol analogs that contain basic substituents are capable of forming a wide variety of salts with various inorganic and organic acids. (See, e.g., U.S. Pat. No. 6,132,740 to Hu (issued Oct. 17, 2000).) Those resorcinol compounds that are acidic in nature are capable of forming base salts with various pharmaceutically acceptable cations. Nonlimiting examples of such salts include the sodium, aluminum, calcium, magnesium, zinc, diethanolammonium, and potassium salts. These salts can be prepared by conventional techniques. (See, e.g., U.S. Pat. No. 6,132,740.) For a review of suitable salts, see, e.g., Berge et al, J. Pharm. Sci. 66:1-19 (1977) and Remington: The Science and Practice of Pharmacy, 20th Ed., ed. A. Gennaro, Lippincott Williams & Wilkins, 2000.

C) Formulation

As noted above, the invention is directed to a formulation for delivering skin lightening agents. The formulation includes at least one skin-lightening agent and a carrier containing at least one hydroxyl solvent and at least one co-solvent. Typically, the formulation will be a liquid. It may be a solution, a suspension, or an emulsion, depending upon the specific components utilized and their relative concentration.

The concentration of the skin-lightening agent (i.e. the active agent) can vary widely. Typically, the active agent will be present in the formulation in the quantity of from about 0.1% (wt/vol) to about 10% (wt/vol). Alternatively, the amount of active agent is from about 0.1% (wt/vol) to about 5% (wt/vol) of the formulation. Alternatively, the amount of active agent in the formulation is from about 0.5% (wt/vol) to about 4% (wt/vol) of the formulation. Alternatively, the amount of active agent in the formulation is from about 2% (wt/vol) to about 4% (wt/vol) of the formulation. Alternatively, the amount of active agent in the formulation is from about 1% (wt/vol) to about 3% (wt/vol) of the formulation. Alternatively, the amount of active agent in the formulation is from about 1% (wt/vol) to about 2% (wt/vol) of the formulation. By way of nonlimiting example, the amount of active agent in the formulation is about 1% (wt/vol), about 2% (wt/vol), about 3% (wt/vol), or about 4% (wt/vol) of the formulation.

The formulation will also contain a carrier. The carrier may comprise up to 99.9% of the formulation. Typically, it will comprise at least 80% of the formulation. The quantity of carrier contained within the formulation will vary with the amount of skin lightening agent utilized and the presence of other active agents, which is described infra.

The carrier will contain at least one hydroxyl compound. The quantity of hydroxyl compound can vary, but will typically comprise from about 25% to about 95% (vol/vol) of the carrier. Typically the carrier will contain from about 50% to about 90% (vol/vol) of the hydroxyl compound. Examples of hydroxyl compounds include glycols, lower alkanols and water. The formulation may contain a single hydroxyl compound or admixtures of different hydroxyl compounds may be used.

In some embodiments, the hydroxyl compound is a glycol. In various embodiments, the glycol is a C₂-C₁₂, C₂-C₁₀, C₂-C₈, or C₂-C₆ glycol. Nonlimiting examples of glycol compounds include methylene glycol, ethylene glycol, propylene glycol, butylene glycol, polyethylene glycol, and hexylene glycol. The glycol compounds may contain chiral centers and therefore may exist in different enantiomeric and diastereomeric forms. Additionally, different regioisomers of the glycol compound may exist. Any regioisomers, any optical isomers and any stereoisomers of these compounds and mixtures thereof, can be used in the formulation of the invention. Other compounds with similar chemical properties can be substituted for the glycol.

The total amount of glycol in the carrier varies from about 0% to about 90% (vol/vol). In various embodiments, the carrier comprises a minimum of about 0%, 5%, 10%, 15%, or 20% glycol (vol/vol). In various embodiments the carrier comprises a maximum of about 90%, 75%, 65%, 60%, 55%, 50%, 45% or 40% (vol/vol)glycol. Any range between any of these upper and lower values is contemplated to be within the scope of the invention.

In some embodiments the carrier comprises more than one glycol compound in order to balance the solubility of the components of the formulation. For example, in some embodiments the carrier contains a mixture of hexylene glycol (“HG”) and propylene glycol (“PG”). Hexylene glycol is also called 2-methyl-2,4-pentanediol, and is available commercially, e.g., from Acros Organics (Morris Plains, N.J.). Propylene glycol is also called 1,2-propanediol, and is available commercially, e.g., from Sigma (St. Louis, Mo.).

In some embodiments, the hydroxyl compound is a lower alkanol. As used herein, the term “lower alkanol” includes all C₁-C₆ alkanols, including without limitation, all straight chain or branched chain or cyclic lower alkanols. In some embodiments, the alkanol is a C₁-C₄ alkanol. Nonlimiting examples of such C₁-C₄ lower alkanols include methanol, ethanol, propanol, isopropanol, butanol, isobutanol, and tert-butanol. In one example the lower alkanol is ethanol (“EtOH”). The alkanol compounds may contain chiral centers and therefore may exist in different enantiomeric and diastereomeric forms. Additionally, different regioisomers of the alkanol compound may exist. Any optical isomers, any regioisomers, and any stereoisomers of these compounds and mixtures thereof, may be used in the formulation of the invention. Lower alkanols can be commercially obtained, e.g., from Fisher Scientific (Pittsburgh, Pa.). Other compounds with similar chemical properties can be substituted for the lower alkanol.

The total amount of lower alkanol in the carrier varies from about 0% to about 90% (vol/vol). In various embodiments, the carrier comprises a minimum of about 0%, 5%, 10%, 15%, or 20% (vol/vol) lower alkanol. In various embodiments the carrier comprises a maximum of about 90%, 75%, 65%, 60%, 55%, 50%, 45% or 40% (vol/vol) lower alkanol. Any range between any of these upper and lower values is contemplated to be within the scope of the invention.

In some embodiments, the hydroxyl compound is water. The amount of water in the carrier varies from about 0% to about 90% (vol/vol). In various embodiments, the carrier comprises a minimum of about 0%, 5%, 10%, 15%, or 20% (vol/vol) water. In various embodiments the carrier comprises a maximum of about 90%, 75%, 65%, 60%, 55%, 50%, 45% or 40% (vol/vol) water. Any range between any of these upper and lower values is contemplated to be within the scope of the invention.

In some embodiments, the carrier comprises a mixture of hydroxyl compounds. For example, in different embodiments the carrier comprises a mixture of at least one glycol and at least one lower alkanol, a mixture of at least one glycol and water, a mixture of at least one lower alkanol and water, or a mixture of at least one glycol, at least one lower alkanol, and water.

The carrier will also contain at least one co-solvent. The co-solvent will be present in the quantity of about 5% to about 50% (vol/vol). More typically it will be present in the quantity of from about 10% to about 30% vol/vol.

In some embodiments, the co-solvent is a dianhydrohexitol analog or derivative thereof. Non-limiting examples of dianhydrohexitols include isosorbide, isomannide, and isoidide. Any such compound, or an analog or derivative thereof can be used in the formulation of the invention. In some embodiments, the co-solvent is a diether derivative of a dianhydrohexitol. In some embodiments, the diether derivative is a dialkyl ether derivative, preferably a di(C₁-C₆)alkyl ether. In some embodiments, the co-solvent is a dialkyl ether derivative of isosorbide, e.g., dimethyl isosorbide (“DMI”), which is available commercially, e.g., from Aldrich (Milwaukee, Wis.).

In some other embodiments, the co-solvent is a lower alkyl ester of a C₁₀-C₂₀ fatty acid. Nonlimiting examples of such fatty acid esters include lower alkyl esters of lauric acid, myristic acid, palmitic acid, stearic acid, and arachidic acid. More specific examples include methyl laurate, methyl palurate, methyl stearite, ethyl laurate, ethyl palmite, ethyl stearite, etc.

In still other embodiments, the co-solvent is a cyclic amide, carbamate, or carbonate. In some embodiments, the co-solvent is a pyrrolidone, including, e.g., an N-alkyl pyrrolidone. One nonlimiting example of such a useful pyrrolidone is N-methyl-2-pyrrolidone.

Any of the co-solvents described above may contain chiral centers and therefore may exist in different enantiomeric and diastereomeric forms. Additionally, different regioisomers of the compound may exist. Any regioisomers, any optical isomers, and any stereoisomers of these compounds and mixtures thereof, can be used as the active agent in the formulation of the invention. Other compounds with similar chemical properties can also be used for the co-solvent.

The ratio of the components in the carrier will vary depending on the solubility and permeability of the skin-lightening agent used in the formulation as well as other factors. In some embodiments, this ratio is varied in order to achieve the best efficacy of the agent without increasing the amount of irritation caused by the formulation. For example, the amount of glycol in the carrier varies from about 0% to about 90% (vol/vol). In some embodiments HG and PG are both components of the carrier, and the amount of HG varies from about 0% (vol/vol) to about 15% (vol/vol), and the amount of PG varies from about 20% (vol/vol) to about 50% (vol/vol). The amount of co-solvent in the carrier varies from about 5% (vol/vol) to about 40% (vol/vol). In some embodiments, the amount of co-solvent in the carrier varies from about 10% (vol/vol) to 20% (vol/vol). The amount of lower alkanol in the carrier varies from about 0% (vol/vol) to about 90% (vol/vol). Although it is possible for the ratios of the components of the carrier to vary depending on the specific active agent used, a useful formulation comprises: about 45% glycol, about 15% co-solvent, and about 40% lower alkanol (vol/vol/vol). A particular useful formulation comprises: about 5% HG, about 40% PG, about 15% DMI, and about 40% ethanol (vol/vol/vol/vol). Additionally, the amount of lower alkanol in the carrier can be varied depending on the amount of skin-lightening agent added to the formulation, while keeping the ratio of the other components of the carrier approximately the same. For example, the lower alkanol concentration may be reduced based on calculations of the amount of skin-lightening agent used depending on the desired concentration

In addition to the skin-lightening agent, one of skill in the art would be aware of numerous other pharmaceutical excipients that could be added to the carrier to enhance its elegance and appeal to the consumer. The CTFA Cosmetic Ingredient Handbook, Second Edition (1992) describes a wide variety of nonlimiting cosmetic and pharmaceutical ingredients commonly used in the skin care industry, which may be suitable for use in the formulation of the present invention.

Nonlimiting examples of these ingredient classes include: aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-caking agents, antifoaming agents, antioxidants, binders, buffering agents, bulking agents, chelating agents, colorants, cosmetic astringents, cosmetic biocides, denaturants, pH adjusters, propellants, reducing agents, sequestrants, skin-conditioning agents (e.g., humectants, including miscellaneous and occlusive), skin soothing and/or healing agents (e.g., panthenol analogs (e.g., ethyl panthenol), aloe vera, pantothenic acid analogs, allantoin, bisabolol, and dipotassium glycyrrhizinate), etc.

Other active ingredients may be added to the formulation. One such example is sunscreens (UVA or UVB blockers). They may be incorporated into the formulation to prevent repigmentation, to protect against sun or UV-induced skin darkening or to enhance the ability to reduce skin melanin and the skin depigmentation action. See, e.g., U.S. Pat. No. 6,132,740 to Hu (issued Oct. 17, 2000). The potential utility of incorporating other active ingredients beyond the skin-lightening agent will be readily apparent to one skilled in the art.

D) Method of Manufacture

The formulation may be prepared using methods well known to those skilled in the art. For example, the formulation is prepared by mixing the carrier components together on a volume to volume or weight to weight basis. The carrier components are combined together in any order. The active agent is weighed out separately and combined with the carrier. In some embodiments, the carrier is added gradually to the dry active agent in order to slowly wet it. Alternatively, the active agent is added to the carrier, which can also be done gradually. In some embodiments, the active agent dissolves in the carrier. To facilitate dissolution, in some embodiments the container holding the carrier and the active agent are stirred by methods including but not limited to vortexing or placing a magnetic stir bar in the container which is placed on a magnetic stirrer.

E) Dosage and Use

In yet another aspect, the invention provides a method for lightening skin or preventing the formation of skin pigmentation. This method comprises administering to an area of a subject's skin an effective amount of a pharmaceutical formulation comprising a skin-lightening agent in an admixture with a pharmaceutically acceptable carrier. The formulation may be administered in one dose or may require multiple doses in order to achieve the desired effect.

In order to be effective, the appropriate dose regimen, the amount of each dose administered, and specific intervals between doses of the active compound will depend upon the particular active compound employed, the condition, size, and age of the patient being treated, the toxicity and half-life of the chosen active agent(s), the presence of other drugs in the patient, the effect desired, and the nature and severity of the disease, disorder or condition being treated. Specifically, the active compound is administered in an amount and at an interval that results in the desired treatment of or improvement in the disorder or condition being treated. The trial dosages would be chosen after consideration of the results of animal studies and the clinical literature. A physician would make these determinations.

For example, an effective dosage and treatment protocol can be determined by conventional means, starting with a low dose in laboratory animals and then increasing the dosage while monitoring the effects, and systematically varying the dosage regimen as well. Where possible, it is desirable first to determine in vitro the cytotoxicity of the compound to the tissue type to be treated, and then in a useful animal model system prior to testing and use in humans. Animal studies, such as mammalian studies, are commonly used to determine the maximal tolerable dose (“MTD”) of a bioactive agent per kilogram weight. Those skilled in the art can extrapolate doses for efficacy and avoidance of toxicity to other species, including humans.

One of ordinary skill in the art will appreciate that the endpoint chosen in a particular case will vary according to the disease, condition, or disorder being treated, the outcome desired by the patient, subject, or treating physician, and other factors. When using the formulation to lighten skin color such as, for example, to reverse hyperpigmentation caused by, for example, inflammation or diseases such as melasma, any one of a number of endpoints can be chosen. Endpoints can be defined subjectively such as, for example, when the subject is simply “satisfied” with the results of the treatment. The endpoint can be determined by the satisfaction of the patient or the treating physician with the results of the treatment. Alternatively, endpoints can be defined objectively. For example, the patient's or subject's skin in the treated area can be compared to a color chart. Treatment can then be terminated when the color of the skin in the treated area is similar in appearance to a color on the chart. Alternatively, the reflectance of the treated skin can be measured, and treatment can be terminated when the treated skin attains a specified reflectance. Alternatively, the melanin content of the treated skin can be measured. Treatment can be terminated when the melanin content of the treated skin reaches a specified value. Melanin content can be determined in any way known to the art, including by histological methods, with or without enhancement by stains for melanin.

Typically however, the skin lightening agent will be present in the formulation in the quantity of 0.1 to 10% (wt/vol). The formulation will be applied to the skin requiring lightening from 1 to 4 times daily. Administration will continue on a daily basis until the desired pigmentation is achieved. Subsequent reapplications may be required on a periodic basis.

There are numerous conditions for which the skin-lightening formulation may be beneficial which would be known to one of skill in the art. See, e.g., Freedberg et al., eds. (1999) Fitzpatrick's Dermatology in General Medicine, Fifth Edition, 987, McGraw-Hill, New York. Such disorders include, but are not limited to, regional hyperpigmentation caused by melanocytic hyperactivity, such as idiopathic melasma occurring either during pregnancy (mask of pregnancy or chloasma) or secondary to estrogen-progesterone contraception; local hyperpigmentation caused by benign melanocytic hyperactivity and proliferation such as lentigo senilis; accidental hyperpigmentation such as post-lesional pigmentation and scarring; postinflammatory hyperpigmentation; and certain forms of leukoderma such as vitiligo where, if the injured skin cannot be repigmented, the residual zones of normal skin are depigmented to impart a homogeneous white color to the entire skin. In addition, there are numerous other conditions for which skin-lightening may be beneficial. By way of nonlimiting example, such skin conditions may be caused by lentigines, Moynahan's syndrome, centrofacial neurodysraphic lentiginosis, Peutz-Jegher syndrome, PUVA, Sotos' syndrome, solar lentigo (such as liver spots), eruptive lentigines, café au lait macules, neurofibromatosis, Albright's syndrome, Silver-Russell syndrome, Westerhof's syndrome, Watson's syndrome, Bloom's syndrome, gastrocutaneous syndrome, Becker's melanosis, nevus spilus, ephelides, NAME/LAMB syndrome, ichthyosis nigricans, porphyria cutanea tarda, hemochromatosis, hepatolenticular degeneration, Gaucher's disease, Niemann-Pick disease, ACTH- and MSH-producing tumors, exogenous ACTH therapy, Addison's disease, estrogen therapy, Carney's complex syndrome, arsenicals, busulfan, photochemical agents (such as psoralens or tar), Berloque dermatitis, Kwashiorkor, pellagra, sprue, vitamin B₁₂ deficiency, ultraviolet radiation (such as radiation tanning or suntanning), thermal radiation, alpha, beta, gamma ionizing radiation, trauma (such as chronic pruritus), postinflammatory melanosis (such as exanthems or drug eruptions), Lichen planus, discoid lupus erythematosus, melanoma, mastocytosis, acanthosis nigricans with adenocarcinoma and lymphoma, systemic scleroderma, chronic hepatic insufficiency, Whipple's syndrome, Cronkhite-Canada syndrome, neurocutaneous melanosis, familial periorbital hyperpigmentation, familial progressive hyperpigmentation, Dowling-Degos disease, dyskeratosis congenita, Fanconi's syndrome, human chimaera, acropigmentation of Dohi, reticulate acropigmentation of Kitamura, dermatopathia pigmentosa reticularis, POEMS syndrome, carbon baby syndrome, systemic 5-fluorouracil, cyclophosphamide, topical nitrogen mustard, Bleomycin, lichen simplex chronicus, atopic dermatitis, psoriasis, and tinea versicolor.

In addition to pharmaceutical uses, the formulations of the current invention are useful for cosmetic purposes. Cosmetic applications for methods of the present invention include the topical application of compositions containing one or more compounds to enhance or otherwise alter the visual appearance of normal skin, which is not affected by a disorder. Occurrences in the skin of, noticeable but undesired pigmentation as a result of melanin production (e.g. freckles) can be treated using the methods of the present invention.

In still yet another aspect, the invention provides for a kit for preparing a skin-lightening formulation. The kit contains a skin-lightening agent, as described above, at least one container, a co-solvent, and at least one hydroxyl compound selected from the group consisting of glycols, lower alkanols, and water. In some embodiments, the co-solvent and hydroxyl compound are premixed to form a carrier. In some embodiments, the carrier comprises a co-solvent, at least one glycol, and a lower alkanol. In certain particular embodiments, the carrier comprises an isosorbide, e.g., dimethyl isosorbide, at least one glycol, e.g., hexylene glycol and/or propylene glycol, and a lower alkanol, e.g., ethanol. The components of the carrier and the active agent(s) may or may not be provided in a preset unit dose or usage amount.

The container has a variety of purposes, including but not limited to preventing contamination, minimizing evaporation or drying, facilitating mixing, or a variety of other purposes known in the art. The container is made of any material suitable for the desired purpose. For example, in some embodiments the kit includes various containers for containing the components of the formulation and combining them, including but not limited to a divided bottle or a divided foil packet; however, in some embodiments the components are contained within a single, undivided container. Additional nonlimiting examples of containers include syringes, boxes, bags, and the like. For example, in some embodiments, the kit includes one container containing a previously-prepared formulation containing the skin-lightening agent as well as the carrier and any additional components as discussed above. Alternatively, the kit includes separate containers containing the skin-lightening agent and the carrier, and at least one of the components of the carrier can be contained in another container. The components of the carrier can all be in separate containers or in containers containing one or more component together as well. For example, the kit can include separate containers containing the skin-lightening agent and at least one of the co-solvent or the hydroxyl compound. Alternatively, the kit can include separate containers containing the skin-lightening agent and each of the co-solvent and the hydroxyl compound. The kit can also include additional containers in which to combine the various components of the formulation, including but not limited to, a container for combining the components of the carrier and a container for combining the carrier with the skin-lightening agent. The agent is mixed together with the carrier in a container. Alternatively, the components of the carrier are mixed together first and then mixed with the active agent. The kit can also include additional components, such as those described above. These additional components can be in separate containers and then mixed with the other components of the formulation. Alternatively, these additional components can be already mixed with one or more of the components of the formulation.

The same kit can be used for lightening skin and can also include an applicator for the skin-lightening formulation. As used herein, the term “applicator” means any instrument with which the formulation can be applied using any method of application. One of skill in the art would be aware of numerous instruments that could serve as applicators for topically administering the formulation. Nonlimiting examples of applicators include a sponge, a pipette, a cotton swab and a brush.

Optionally, the kit further contains a label indicating that the kit is to be used for lightening skin. The label may also contain instructions directing the procedure for combining the components if necessary and/or the use of such components for lightening skin. The kit may also optionally further contain a package insert indicating that the kit is to be used for lightening skin. The package insert may also optionally contain instructions directing the procedure for combining the components if necessary and/or the use of such components for lightening skin.

It may be desirable to provide a written memory aid, where the written memory aid is of the type containing information and/or instructions for the physician, pharmacist or subject. Nonlimiting examples of such a written memory aid include numbers on the containers corresponding with the days of the regimen on which the formulation should be applied, a card which contains the same type of information, or a calendar printed on a card e.g., as follows “First Week, Monday, Tuesday,” . . . etc. . . . “Second Week, Monday, Tuesday, . . . ” etc. Other variations of memory aids will be readily apparent.

The kit may also optionally include a dispenser designed to dispense the daily doses one at a time. A “daily dose” can be an application of the formulation or several applications of the formulation on a given day. In some cases, the dispenser is equipped with a memory-aid, so as to further facilitate compliance with the regimen. A nonlimiting example of such a memory-aid is a mechanical counter that indicates the number of daily doses that have been dispensed. Another nonlimiting example of such a memory-aid is a battery-powered micro-chip memory coupled with a liquid crystal readout, or audible reminder signal which, for example, reads out the date that the last daily dose has been administered and/or reminds one when the next dose is to be administered.

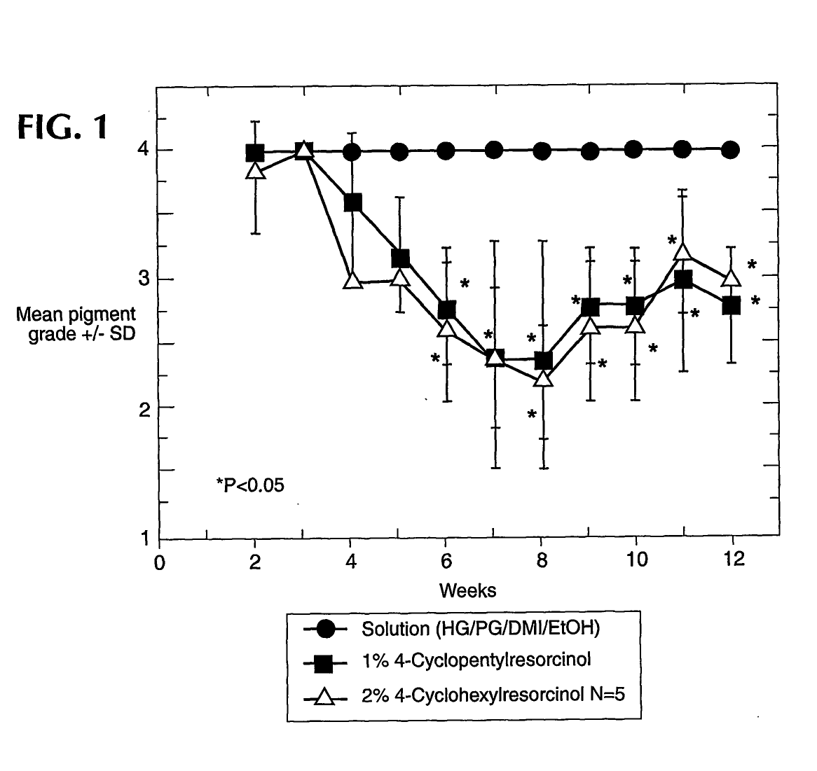

The following nonlimiting examples further illustrate certain preferred embodiments of the present invention. The formulations were tested experimentally on Yucatan swine with dark skin. The Yucatan pigskin shares many physiologic and morphologic characteristics with human skin. For example, as described in U.S. Pat. No. 5,470,567, the thickness and general morphology of epidermis and dermis, tritiated thymidine labeling pattern and index of epidermal cells, epidermal cell turnover time, as well as size, orientation, and distribution of vessels in skin are similar to that in humans. Varying concentrations of 4-cyclopentylresorcinol and 4-cyclohexylresorcinol were administered to the back of Yucatan swine using various carriers. Representative data from the results of these experiments are shown in FIGS. 1-5.

### EXAMPLE 1

### Yucatan Pig

A. Animals

Yucatan swine with dark skin were used in this study. Swine were obtained from Charles River Laboratories, Windham, Me., and Sinclair Laboratories, MO.

B. Preparation of Skin-Lightening Agents and Formulations

4-cyclopentylresorcinol and 4-cyclohexylresorcinol were synthesized and provided by Medicinal Chemistry OSIP/Birmingham, UK. All test materials were prepared by the Department of Pharmaceutical Development/OSI Pharmaceuticals, Inc./Birmingham, UK and stored in the freezer at −20° C.

The solvents and other components used are commercially available and were obtained commercially from the following suppliers: ethanol (absolute) from Fisher (Pittsburgh, Pa.); propylene glycol (USP) from Sigma (St. Louis, Mo.); hexylene glycol from Acros Organics (Morris Plains, N.J.); dimethyl isosorbide from Aldrich (Milwaukee, Wis.); and IPM from BUFA (Netherlands).

Test carriers, also called “vehicles” herein, that were combinations of different solvent components were chosen based on the solubility of the skin-lightening agent in the individual solvents. Key factors when determining the test vehicle were the solubility of the skin-lightening agent in the vehicle, lack of precipitation of the skin-lightening agent in the vehicle, stability of the skin-lightening agent in the vehicle, and penetration of the skin by the skin-lightening agent in the vehicle.

A HG/PG/DMI/EtOH carrier was prepared by mixing together the four solvents on a volume to volume basis in the appropriate ratio. The solvents are mixed together in any order. To combine this carrier with the active agent, the carrier was gradually added to the dry active agent in order to slowly wet the dry material. The active agent then dissolved in the carrier by mixing.

Amounts of solution are measured by weight or volume. Either measurement can be used, but it is important to be consistent in the way the measurements are performed.

The solution of 1% 4-cyclopentylresorcinol in HG/PG/DMI/EtOH was prepared by weighing 0.40239 g of 4-cyclopentylresorcinol and adding 40 ml HG/PG/DMI/EtOH (5.1/2.2/15.4/19.5) (vol/vol/vol/vol) to yield 10.1 mg/ml of formulation solution.

The 2% 4-cyclohexylresorcinol solution in HG/PG/DMI/EtOH was prepared by weighing 0.80307 g 4-cyclohexylresorcinol, and adding 40 ml HG/PG/DMI/EtOH (5.1/2.2/15.4/19.5) (v/v/v/v) to yield 20.1 mg/ml formulation.

Additional formulations with varying concentrations of active agent or other ingredients may be made as described above or as further described herein with modifications in the amounts used to produce formulations with the appropriate concentrations.

C. Methods of Treatment and Evaluating Results

Seven sites measuring approximately 3 cm×3 cm were marked on each side of the animal's spine, between the shoulder blade and hipbone. The sites were far enough apart so that there was no cross-over. Multiple treatments were performed on the back of each animal. Each animal in the experiment received the same treatments.

Each spot was topically treated with 20 μl of test solution applied twice daily, five days per week. This amounts to the same dose of active agent. Solutions were administered by pipetting the exact volume to the designated area with a micropipette and then spreading the solution. Each spot or test site on the animal's skin was graded 3 times a week on a scale of 0 to 4 for pigment change, erythema and scaling. Treatments were continued until pigmentation was reduced to a grade 1 level (marked uniform decrease in pigmentation) or for up to 12 weeks.

Results were analyzed using different types of statistical analysis, ANOVA, Kruskal-Wallis test and Dunnett's multiple comparison, as indicated below. ANOVA is a statistical test that assumes sampled data is from populations that follow a Gaussian bell-shaped distribution and which works well, especially with large numbers, even if the distribution is only approximately Gaussian. The Kruskal-Wallis test is a nonparametric test that compares three or more unpaired groups. The Dunnett's test is a procedure for comparing each experimental mean with the control mean. For each graph presented, it is indicated which statistical tests were performed.

D. Testing

The carriers, also called “vehicles” herein, were tested using two different skin-lightening agents. One vehicle tested was dimethyl isosorbide/hexylene glycol/propylene glycol/ethanol. The results are shown in Table 1 and in FIG. 1.

TABLE 1

| | Test material | | Mean pigment |
|---|---|---|---|
| | concentration | | Grade +/− SD¹ |
| Active Agent | (% vol/vol) | Vehicle | (Week 12) |
| | | DMI/HG/ | 4 +/− 0 |
| | | PG/EtOH² | |
| | | (15/5/40/35) | |
| 4-cyclopentylresorcinol | 1% | DMI/HG/ | 2.8 +/− 0.4* |
| | | PG/EtOH² | |
| 4-cyclohexylresorcinol | 2% | DMI/HG/ | 3 +/− 0* |
| | | PG/EtOH² | |

*p < 0.05, statistically different from the respective vehicle control using Kruskal-Wallis test.
¹graded on a scale of 0 to 4 in which 4 = normal skin color; 3 = slight decrease in pigmentation; 2 = definite decrease in pigmentation; 1 = marked uniform decrease in pigmentation; and 0 = complete depigmentation.
²Dimethyl isosorbide/hexylene glycol/propylene glycol/ethanol

FIG. 1 compares the depigmenting effect on Yucatan swine of topically applied 1% (wt/vol) 4-cyclopentylresorcinol, 2% (wt/vol) 4-cyclohexylresorcinol in HG/PG/DMI/EtOH, and the HG/PG/DMI/EtOH solution alone. This figure shows that both active agents have increased efficacy in HG/DMI/PG/EtOH. The data regarding the use of these skin-lightening agents in PG/EtOH is presented in Table 2 below.

No evidence of local skin irritation was apparent over any of the treated sites.

E. Repigmentation

Pigmentation was monitored for 6 weeks after discontinuation of treatment following 12 weeks of topical treatment with 1% 4-cyclopentylresorcinol and 2% 4-cyclohexylresorcinol in HG/DMI/PG/EtOH (5/15/40/40).

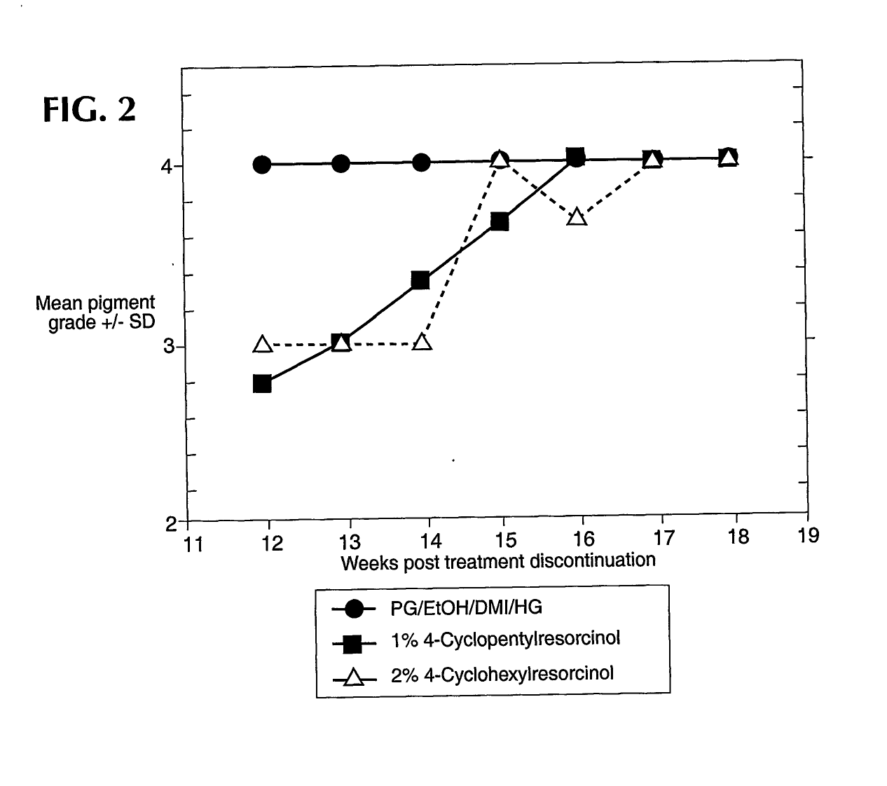

FIG. 2 compares the reversibility of depigmentation after treatment discontinuation for the HG/PG/DMI/EtOH solution alone to treatment with different percentages of 4-cyclopentylresorcinol and 4-cyclohexylresorcinol in the solution. The results show that the test sites treated with 1% 4-cyclopentylresorcinol and 2% 4-cyclohexylresorcinol show signs of gradual repigmentation during 6 weeks after discontinuation, of treatment. Thus, the depigmenting effect was not permanent.

F. Collated Results

The results from several sets of experiments were collated to provide a more comprehensive comparison of the efficacy of 4-cyclopentylresorcinol and 4-cyclohexylresorcinol in different vehicles. Additional vehicles tested that are included in this compilation include: 1) propylene glycol/ethanol (PG/EtOH), which was prepared on a volume to volume basis, to which the active agent was added on a weight to volume basis and vortexed until in solution, and 2) HG/PG/DMI/EtOH+2% (wt/wt) isopropyl myristate (“IPM”), which was prepared by combining HG/PG/DMI/EtOH/IPM in a ratio of 5/40/15/36/2 (wt/wt/wt/wt/wt) to which the active agent was added on a weight to volume basis. IPM was added as a penetration enhancer to determine whether it increased the efficacy of the formulation. The compiled results are presented in Table 2.

TABLE 2

| | Active Agent (mean pigment grade +/− SD)^{1,2} | | | | |
|---|---|---|---|---|---|
| | 1% 4- | 2% 4- | 2% 4- | 4% 4- | 5% 4- |
| Vehicle | cyclopentylresorcinol | cyclopentylresorcinol | cyclohexylresorcinol | cyclohexylresorcinol | cyclohexylresorcinol³ |
| PG/EtOH (3/7) | 3.3 +/− 0.7 | 2.4 +/− 0.9* | 3.0 +/− 0.8 | ND | 3.0 +/− 0* |
| | (12) | (18) | (3) | | (3) |
| HG/PG/DMI/EtOH | 2.8 +/−0.6* | 2.0 +/− 0.7* | 2.8 +/− 0.6* | ND | ND |
| (5/40/15/40) | (10) | (5) | (10) | | |
| HG/PG/DMI/EtOH + | 2.0 +/− 1.2* | 1.4 +/− 0.9* | 2.4 +/− 1.1* | ND | ND |
| 2% IPM | (5) | (5) | (5) | | |

¹Graded on a scale of 0 to 4, 0 = complete depigmentation, 1 = marked even reduction in pigmentation, 2 = moderate even reduction in pigmentation, 3 = slight uneven reduction in pigmentation and 4 = normal skin color
²Numbers in () show number of animals used
*p < 0.05, statistically different from the respective vehicle systems which showed no evidence of depigmentation (grade = 4.0)
³Vehicle was PG/EtOH (5/5)

The Kruskall-Wallis Test and Dunnetts multiple comparison test were used to calculate these results based on the compilation of data from several different experiments using the same protocol.

These formulations were all prepared substantially as described above; however, the amounts were modified to produce the appropriate concentrations.

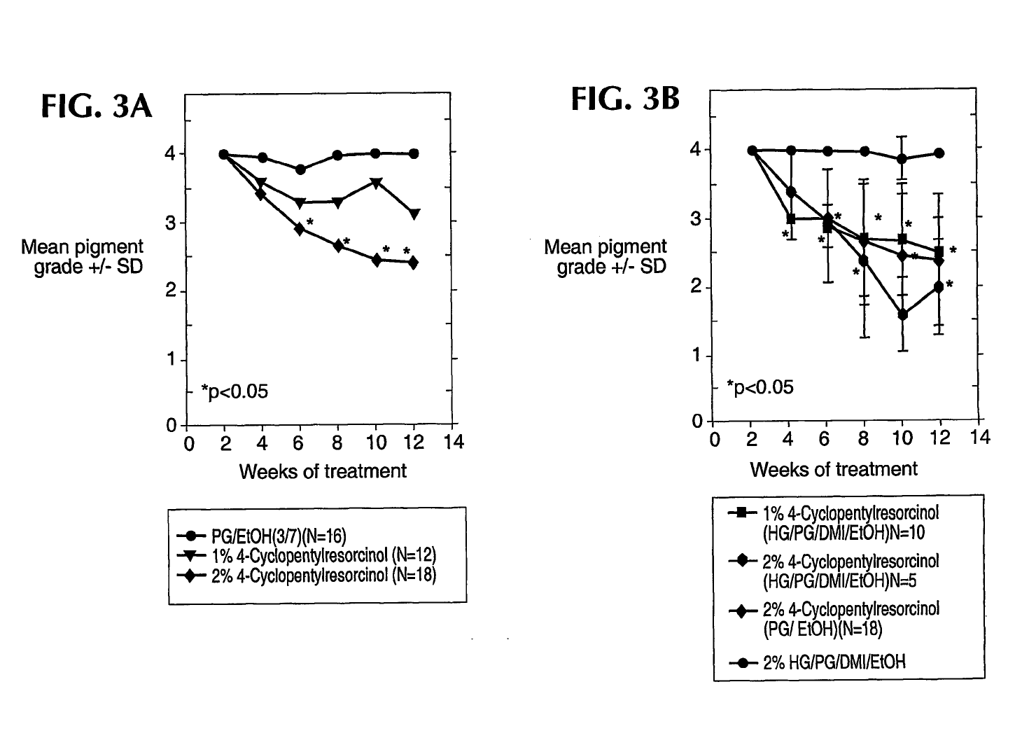

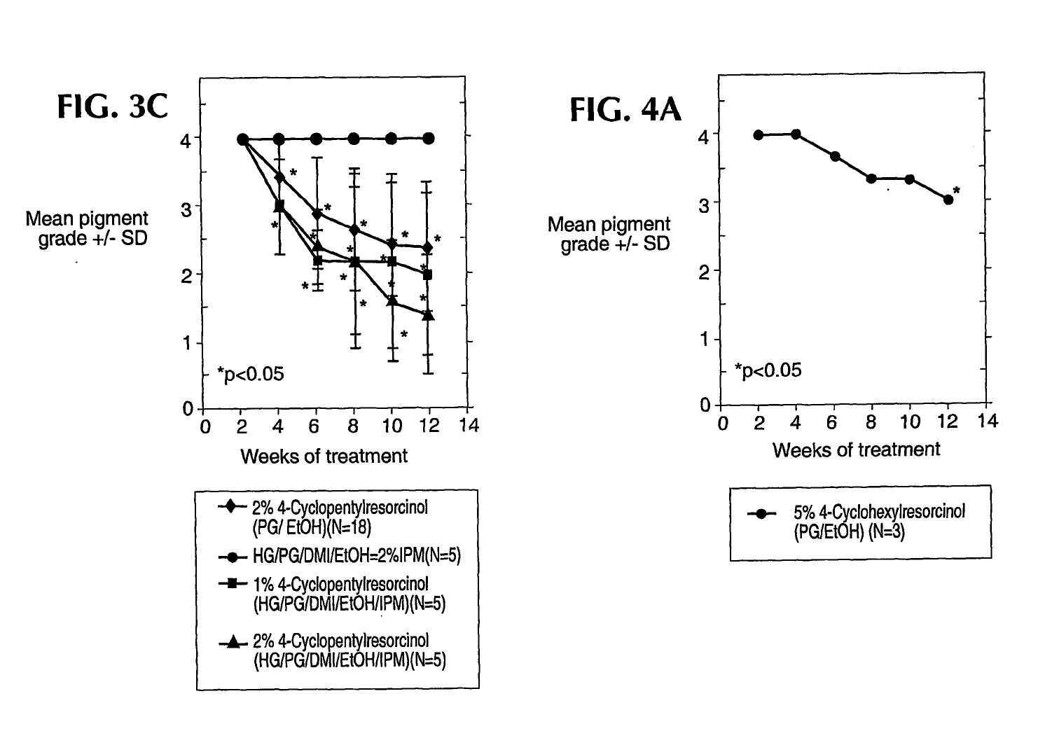

FIG. 3 shows the comparative effect of topically applied 4-cyclopentylresorcinol on Yucatan swine pigmentation in three different vehicle systems. The data was analyzed using a nonparametric method (the Kruskal-Wallis test and Dunnett's multiple comparison).

FIG. 3A compares the effect on the skin pigmentation of Yucatan swine of topical administration of 1% (wt/vol) and 2% (wt/vol) 4-cyclopentylresorcinol in PG/EtOH (30/70) to vehicle alone.

FIG. 3B shows a comparison of 4-cyclopentylresorcinol in HG/PG/DMI/EtOH (5/40/15/40) at 1% (wt/vol) and 2% (wt/vol) concentrations compared to 2% (wt/vol) 4-cyclopentylresorcinol in PG/EtOH and to the HG/PG/DMI/EtOH vehicle alone.

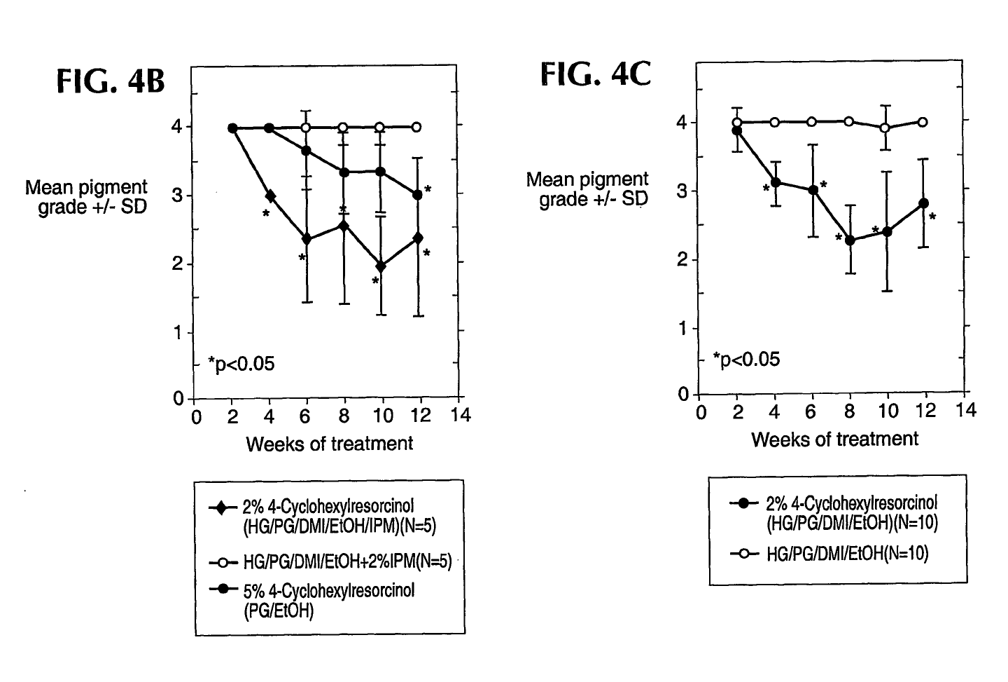

FIG. 3C shows the effects of adding 2% (wt/wt) IPM to the HG/PG/DMI/EtOH vehicle. FIG. 4C also compares the effect on the skin pigmentation of Yucatan swine of topical administration of 1% (wt/vol) and 2% (wt/vol) 4-cyclopentylresorcinol in HG/PG/DMI/EtOH/IPM to 2% (wt/vol) 4-cyclopentylresorcinol in PG/EtOH and to the HG/PG/DMI/EtOH plus 2% (wt/wt) IPM vehicle alone.

The results show that in HG/PG/DMI/EtOH, 1% and 2% 4-cyclopentylresorcinol are statistically different from vehicle and show a trend in the direction of reduced pigmentation but are not statistically different from the results using the PG/EtOH vehicle. No irritation was apparent.

FIG. 4 shows the comparative effect of topical administration of 4-cyclohexylresorcinol on Yucatan swine pigmentation in three different vehicle systems. The data was analyzed using a nonparametric method (the Kruskal-Wallis test and Dunnett's multiple comparison).

FIG. 4A shows the effect on the skin pigmentation of 5% (wt/vol) 4-cyclohexylresorcinol in PG/EtOH.

FIG. 4B compares the effect on the skin pigmentation of 2% (wt/vol) 4-cyclohexylresorcinol in HG/PG/DMI/EtOH/IPM to the HG/PG/DMI/EtOH plus 2% (wt/wt) IPM solution alone and to 5% (wt/vol) 4-cyclohexylresorcinol in PG/EtOH.

FIG. 4C compares the effect on the skin pigmentation of 2% (wt/vol) 4-cyclohexylresorcinol in HG/PG/DMI/EtOH to the HG/PG/DMI/EtOH solution alone.

The results shown in these graphs indicate HG/PG/DMI/EtOH with or without 2% (wt/wt) IPM improved the depigmenting action of 2% 4-cyclohexylresorcinol. No irritation was apparent.

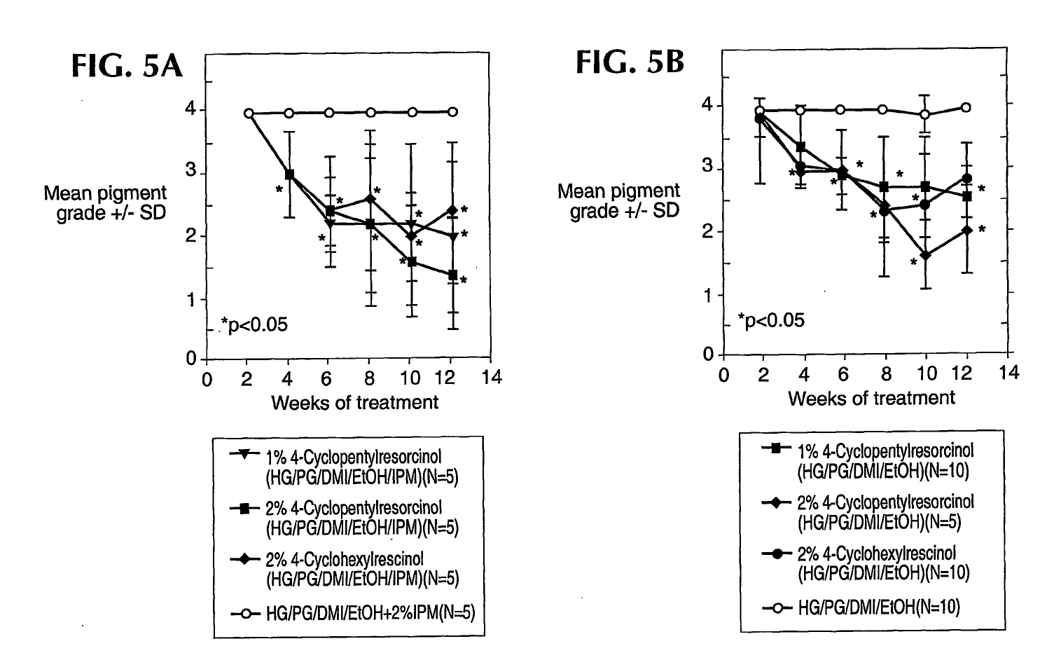

FIG. 5 shows a comparison of the effect of the topical administration of 4-cyclopentylresorcinol and 4-cyclohexylresorcinol in two different vehicles on skin pigmentation in Yucatan swine. The data was analyzed using a nonparametric method (the Kruskal-Wallis test and Dunnett's multiple comparison).

FIG. 5A compares the effect on the skin pigmentation of 1% (wt/vol) and 2% (wt/vol) 4-cyclopentylresorcinol in an HG/PG/DMI/EtOH/IPM vehicle and 2% (wt/vol) 4-cyclohexylresorcinol in an HG/PG/DMI/EtOH/IPM vehicle to the HG/PG/DMI/EtOH plus 2% (wt/wt) IPM vehicle alone.

FIG. 5B compares the effect on the skin pigmentation of 1% (wt/vol) and 2% (wt/vol) 4-cyclopentylresorcinol in a HG/PG/DMI/EtOH vehicle and 2% (wt/vol) 4-cyclohexylresorcinol in the HG/PG/DMI/EtOH vehicle to the HG/PG/DMI/EtOH vehicle alone. No irritation was apparent.

Overall, the results in these Figures indicate that the HG/PG/DMI/EtOH vehicle improved the depigmentation efficacy of both 4-cyclopentylresorcinol and 4-cyclohexylresorcinol in Yucatan pigs. No irritation was observed for those formulations using HG/PG/DMI/EtOH as a vehicle:

### Equivalents

While the foregoing invention has been described in some detail for purposes of clarity and understanding, it will be appreciated by one skilled in the art from a reading of this disclosure that various changes in form and detail can be made without departing from the scope of the invention and the attached claims.

1. A pharmaceutical formulation for topically administering a skin-lightening agent, the formulation comprising a skin-lightening agent which said skin lightening agent is a 4-substituted resorcinol which is in admixture with a pharmaceutically acceptable carrier, the carrier comprising a co-solvent and at least one hydroxyl compound selected from the group consisting of glycols, lower alkanols and water.

2. The formulation according to claim 2 in which said 4-substituted resorcinol is selected from the group consisting of 4-cyclohexyl resorcinol and 4-cyclopentyl resorcinol.

3. The formulation according to claim 3 in which said carrier is comprised of a glycol and a lower alkanol.

4. The formulation according to claim 4 in which glycol is selected from the group consisting of methylene glycol, ethylene glycol, propylene glycol, butylenes glycol, polyethylene glycol, and hexylene glycol.

5. The formulation according to claim 4 in which said lower alkanol is selected from the group consisting of methanol, ethanol, propanol, isopropanol, butanol, isobutanol, and tert-butanol.

6. The formulation according to claim 4 in which said co-solvent is a dianhydrohexitol analog or derivative thereof.

7. The formulation according to claim 2 in which said carrier is comprised of: 1) from about 25 to about 95% (vol/vol) of a hydroxyl compound selected from the group consisting of glycol, and a lower alkanol; and 2) about 5 to about 50% (vol/vol) of a co-solvent which is a dianhydrohexitol analog or derivative thereof.

8. The formulation according to claim 8 in which said carrier is an admixture of hexylene glycol, propylene glycol, ethanol, and dimethyl isorbide.

9. A pharmaceutical formulation for topically administering a skin-lightening agent in which:
a) the skin lightening agent is 4-cyclopentyl resorcinol which is in admixture with a carrier,
b) said carrier is comprised of
a. about 15% (vol/vol) of dimethyl isorbide,
b. about 5% (vol/vol) of hexylene glycol,
c. about 40% (vol/vol) of propylene glycol and,
d. about 35% (vol/vol) of ethanol.

10. (canceled)

11. Use of a formulation according to claim 1 in the manufacture of a medicament for lightening skin.

12. Use of a formulation according to claim 2 in the manufacture of a medicament for lightening skin.

13. Use of a formulation according to claim 3 in the manufacture of a medicament for lightening skin.

14. Use of a formulation according to claim 4 in the manufacture of a medicament for lightening skin.

15. Use of a formulation according to claim 5 in the manufacture of a medicament for lightening skin.

16. Use of a formulation according to claim 6 in the manufacture of a medicament for lightening skin.

17. Use of a formulation according to claim 7 in the manufacture of a medicament for lightening skin.

18. Use of a formulation according to claim 8 in the manufacture of a medicament for lightening skin.

19. Use of a formulation according to claim 9 in the manufacture of a medicament for lightening skin.
